SEQUENCE LISTING (24 sequences)

SEQ ID NO: 1 - protein, 6 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWRRWI

SEQ ID NO: 2 - protein, 6 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWRRWI

SEQ ID NO: 3 - protein, 6 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWRRWV

SEQ ID NO: 4 - protein, 6 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWRRWV

SEQ ID NO: 5 - protein, 6 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWIKWR

SEQ ID NO: 6 - protein, 6 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWIKWR

SEQ ID NO: 7 - protein, 6 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWVKWI

SEQ ID NO: 8 - protein, 6 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWVKWI

SEQ ID NO: 9 - protein, 6 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWIKWI

SEQ ID NO: 10 - protein, 6 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWIKWI

SEQ ID NO: 11 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWIKWIKWI

SEQ ID NO: 12 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWIKWIKWI

SEQ ID NO: 13 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KFIKFIKFI

SEQ ID NO: 14 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KFIKFIKFI

SEQ ID NO: 15 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWRRWVRWI

SEQ ID NO: 16 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWRRWVRWI

SEQ ID NO: 17 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
IWRVWRRWK

SEQ ID NO: 18 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
IWRVWRRWK

SEQ ID NO: 19 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KFRRFVRFI

SEQ ID NO: 20 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KFRRFVRFI

SEQ ID NO: 21 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KPRRPVRPI

SEQ ID NO: 22 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KPRRPVRPI

SEQ ID NO: 23 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWIRWVRWI

SEQ ID NO: 24 - protein, 9 aa, Artificial
  Artificial peptide performed by solid phase peptide synthesis using standard Fmoc (N-(9-fluoroenyl) methoxycarbonyl) chemistry manually on PAL resin (5-(4-Fmoc-aminomethyl-3,5-dimethoxyphenoxy)-valeric acid-MBHA
KWIRWVRWI

(19) United States
(12) Patent Application Publication
(10) Pub. No.: US 2004/0072990 A1
(43) Pub. Date: Apr. 15, 2004

(54) Antimicrobial peptides with reduced hemolysis and methods of their use

(76) Inventors: Shiou-Ru Tzeng, Brunaby (CA); Jya-Wei Cheng, Brunaby (CA)

Correspondence Address: ROSENBERG, KLEIN & LEE, 3458 ELLICOTT CENTER DRIVE-SUITE 101, ELLICOTT CITY, MD, 21043, US

(21) Appl. No.: 10/247,476

(22) Filed: Sep. 20, 2002

(51) Int. Cl. A61K 38/14; A61K 38/12; C07K 7/64

(52) U.S. Cl. 530/317; 514/9

(57) ABSTRACT

The invention is directed to antimicrobial peptides related to cyclic and short peptides (less than 10 amino acid residues) with unique patterns of aromatic and cationic residues that perform a wide range of antimicrobial activities but display low hemolysis.

### FIELD OF THE INVENTION

The invention related to the field of antibiotic peptides. In particular, the invention is directed to cyclic and short peptides (less than 10 amino acid residues) with unique patterns of aromatic and cationic residues that have a wide range of antimicrobial activities. These extra compact peptides of the invention perform brilliant efficacy and low hemolytic action as compared to other long peptides with aromatic and cationic residues.

### BACKGROUND AND THE INVENTION

The emergence of bacterial strains that are resistant to conventional antibiotics has prompted a search for new therapeutic agents, including various antimicrobial peptides of animal origin. Antimicrobial peptides have been recognized to play important roles in the innate host defense mechanisms of most living organisms including plants, insects, amphibians and mammals, and are known to possess potent antibiotic activity against bacteria, fungi, and even certain viruses. The antimicrobial peptides readily partition into phospholipid bilayers with a fraction of >95% bound to lipid and compromise membrane integrity. They are able to form small, transient conductance increases in planar lipid bilayers and partially depolarize the cytoplasmic membrane potential gradient of bacteria. The protective function of antimicrobial peptides in host defense has been convincingly demonstrated in Drosophila, where their reduced expression dramatically decreases survival after microbial challenge. In mammals, this function is suggested by defective bacterial killing in the lung of cystic fibrosis (CF) patients and in the small mice. The antimicrobial peptides found in mammals are classified into the cysteine-rich defensins (α- and β-defensin) and various cathelicidin families. Cathelicidin family contain a highly conserved signal sequence and proregion (“cathelin”) and a variable antibacterial sequence in the C-terminal domain. Many of the cathelicidins contain a characteristic elastase cleavage site between the anionic cathelin domain and the cationic C-terminal peptide domain. Proteolytic processing at this site has been observed in bovine and porcine neutrophils and was required for microbicidal activity. Based on the amino acid composition and structure, the cathelicidin family is classified three groups. The first group contains the amphipathic α-helical peptides such as LL-37, CRAMP, SMAP-29, PMAP-37, BMAP-27, and BMAP-28. The second group contains the Arg/Pro-rich or Trp-rich peptides including Bac5, Bac7, PR-39, and indolicidin. The third group includes Cys-containing peptides such as protegrins. Since antimicrobial peptides are generally low molecular mass molecules (<5 kDa) possessing broad-spectrum activities and constituting an important part of the host defense against microbial infections, they provide a starting point for designing low molecular of antibiotic compounds. Furthermore, they are known to have a propensity to fold into amphipathic structures with clusters of hydrophobic and charge regions, a feature closely related to their membranolytic activity. Although often displaying broad-spectrum antimicrobial activity, the peptides are, to varying degrees, hemolytic against human erythrocytes which severely limits their therapeutic potential. This task was primarily aimed to design antimicrobial peptides with the activities against clinically important bacterial strains by synthetic modifications of its primary structure and, secondarily, to obtain some information about important structural features of the peptide. Our results have shown that it is possible to improve the activities, or reduce the toxicities, of naturally occurring peptide antibiotics by producing synthetic analogs with modified primary and/or secondary structures. The invention relates generally to antibiotics and it concerns about novel broad-spectrum antimicrobial peptides containing a tryptophan-rich sequence and their derivatives which exhibit antimicrobial activities but with low hemolytic performances. Briefly, the present invention is directed to design antibiotic peptides with broad-spectrum of antimicrobial activities against Gram-positive and Gramnegative bacteria, protozoa, fungi, and the enveloped virus HIV-1.

### SUMMARY OF THE INVENTION

The present invention is directed to design cyclic and short peptides with improved serum compatibility and reduced hemolytic activities. Furthermore, the peptides of the invention exhibit broad-spectrum antimicrobial activities against Gram-positive, Gram-negative bacteria and multi-drug-resistant pathogens. The antimicrobial peptides of this invention is generally composed of less than 10 amino acids residues and comprising the amino acid sequence:

(A₁X₁A₂)_{N}

wherein:

A₁ represents Arginine, Lysine, Valine, or Isolecine

X₁ represents Trptophan, Phenylalanine, or Proline

A₂ represents Arginine, Lysine, Valine, or Isolecine

N represents 1, 2 or 3

Topology represents cyclic or linear.

The invention provides several advantages. First, the peptides of the invention are less than 10 amino acid residues and extremely compact so that it is effective to span the cell membrane with relatively few amino acids. Secondly, the best peptides from the invention perform greatly broad-spectrum activity against antibiotic resistant bacteria, combined with activity against the medically important fungus. In addition, these peptides possess the anti-endotoxin activity and work synergistically with traditional antibiotics. The most important of the invention offers improved serum compatibility and performs extremely low hemolysis against human red blood cells as compared with the naturally occurring protegrins and indolicidin analogs.

### BRIEF DESCRIPTION OF THE FIGURES

FIG. 1 A survival curve for (A) Bacillus. Substilis ATCC6633; (B) Staphylococcus aureus ATCC9144; (C) E. coli ATCC25922; (D) Pseudomonas aeruginosa ATCC29213 treated with Pac-525 (solid circle) and Pac-524 (open circle).

FIG. 2 Pac-525 induced inner membrane permeabilization and assessed by NPN uptake assay (0 μg/ml: solid square; 1 μg/ml: open circle; 2 μg/ml: open square; 3 μg/ml: open square).

FIG. 3 Tryptophan fluorescence reports SDS-induced changes local environment of Pac-525.

FIG. 4 Circular dichroism spectra of (A) Pac-525 and (B) Pac-526 were shown in phosphate buffer (open circle) or in 10 mM SDS (solid circle).

FIG. 5 The figure showed the extremely low hemolysis against human red blood cells. (solid circles: melittin; open circle: Pac-525; solid triangle:

Pac-527).

### DETAILED DESCRIPTION OF THE INVENTION

Like the trytophan-rich peptides as described in U.S. Pat No. 6,303,575; U.S. Pat No. 6,180,604; U.S. Pat No. 5,821,224; U.S. Pat No. 5,547,939; U.S. Pat No. 5,534,939; U.S. Pat No. 5,459,325, and U.S. Pat No. 5,324,716, all of naturally occurring peptides having the amino acid sequence are longer than 10 residues and undergo rapid proteolysis in vivo. Indolicidin analogues have the general amino acid sequence I-L-P-W-K-W-P-W-W-P-W-X, where X represents 1 or 2 selected amino acids (U.S. Pat No. 5,534,939). These previously described trptophan-rich peptides are distinguishable from those of the present invention in that our design of antimicrobial peptides antimicrobial peptides is directed to cylic and short peptides (less than 10 amino acids) with broad-spectrum microbicidal activities, improved selectivity and low hemolysis. Cyclization of linear antimicrobial peptides may have several advantages with regard to selectivity and stability, for example: (i) Unfolded peptides may form aggregates because of hydrophobic interactions, leading to nonspecific adsorption to normal mammalian cells and low solubility. Placing constraints on their unfolded conformations and thus restricting exposure of hydrophobic stretches of amino acids can limit the hydrophobic interactions. Furthermore, these constraints can enhance the role of electrostatic interactions in initial binding with the negatively charged target membrane, thus substantially increasing selectivity toward bacteria versus mammalian cells. (ii) To be bound and cleaved by protease, a peptide must present the cleavage site in an extended structure. Thus, cyclization of short peptides may limit their accessibility to protease activity due to their rigid and constrained structure. (iii) Since cyclization seems to affect activity only towards Gram-negative bacteria, further studies along this line may assist in the design of bacteria-specific lytic peptides. The peptides of the invention can be described by the following formulas:

(A₁X₁A₂)_{N}

wherein:

A₁ represents Arginine, Lysine, Valine, or Isolecine

X₁ represents Trptophan, Phenylalanine, or Proline

A₂ represents Arginine, Lysine, Valine, or Isolecine

N represents 1, 2 or 3

Topology represents cyclic or linear

Examples of the microbicidal peptides from the invention:

Cyclo-K F I

Linear-K F I

Cyclo-R F I

Linear-R F I

Cyclo-R F V

Linear-R F V

Cyclo-K F R

Linear-K F R

Cyclo-K W V

Linear-K W V

Cyclo-K W I

Linear-K W I

Cyclo-K W R

Linear-K W R

Cyclo-R W V

Linear-R W V

Cyclo-K W R R W I

Linear-K W R R W I

Cyclo-K W R R W V

Linear-K W R R W V

Cyclo-K W I K W R

Linear-K W I K W R

Cyclo-K W V K W I

Linear-K W V K W I

Cyclo-K W I K W I

Linear-K W I K W I

Cyclo-K W I K W I K W I

Linear-K W I K W I K W I

Cyclo-K F I K F I K F I

Linear-K F I K F I K F I

Cyclo-K W R R W V R W I

Linear-K W R R W V R W I

Cyclo-I W R V W R R W K

Linear-I W R V W R R W K

Cyclo-K F R R F V R F I

Linear-K F R R F V R F I

Cyclo-K P R R P V R P I

Linear-K P R R P V R P I

Cyclo-K W I R W V R W I

Linear-K W I R W V R W I

### EXAMPLE 1

### Design, Synthesis, Purification and Characterization of Peptides

Peptides analogues and their names are listed in Table 1. All of the amino acids are denoted by the one-letter amino acid code.

TABLE 1

| Name | amino acid sequence | |
|---|---|---|
| Pac 301 C | K W R R W I | (SEQ ID NO:1) |
| Pac 301 L | K W R R W I | (SEQ ID NO:1) |
| Pac 302 C | K W R R W V | (SEQ ID NO:2) |
| Pac 302 L | K W R R W V | (SEQ ID NO:2) |
| Pac 303 C | K W I K W R | (SEQ ID NO:3) |
| Pac 303 L | K W I K W R | (SEQ ID NO:3) |
| Pac 304 C | K W V K W I | (SEQ ID NO:4) |
| Pac 304 L | K W V K W I | (SEQ ID NO:4) |
| Pac-305 C | K W I K W I | (SEQ ID NO:5) |
| Pac-305 L | K W I K W I | (SEQ ID NO:5) |
| Pac-521 C | K W I K W I K W I | (SEQ ID NO:6) |
| Pac-521 L | K W I K W I K W I | (SEQ ID NO:6) |
| Pac-522 C | K F I K F I K F I | (SEQ ID NO:7) |
| Pac-522 L | K F I K F I K F I | (SEQ ID NO:7) |
| Pac-525 C | K W R R W V R W I | (SEQ ID NO:8) |
| Pac-525 L | K W R R W V R W I | (SEQ ID NO:8) |
| Pac-526 C | I W R V W R R W K | (SEQ ID NO:9) |
| Pac-526 L | I W R V W R R W K | (SEQ ID NO:9) |
| Pac-527 C | K F R R F V R F I | (SEQ ID NO:10) |
| Pac-527 L | K F R R F V R F I | (SEQ ID NO:10) |
| Pac-528 C | K P R R P V R P I | (SEQ ID NO:11) |
| Pac-528 L | K P R R P V R P I | (SEQ ID NO:11) |
| Pac-529 C | K W I R W V R W I | (SEQ ID NO:12) |
| Pac-529 L | K W I R W V R W I | (SEQ ID NO:12) |

C: cyclic form;
L: linear form.

ALL linear peptides were performed by solid phase peptide synthesis using standard Fmoc (N-(9-fluoroenyl)methoxycarbonyl) Chemistry manually on PAL resin (5-(4-Fmoc-aminomethyl-3,5-dimethoxyphenoxy)-valeric acid-MBHA resin). The on-resin Fmoc protecting group was removed by 20% piperidine/DMF. The coupling reaction was about 1˜1.5 hours and checked by the ninhydrin test. The crude peptide was cleaved from the resin by mixing with 95% TFA cleavage mixture for 1 to 1.5 hours. The crude peptide was purified by reverse phase high-pressure liquid chromatography. The column for RP-HPLC purification was a semi-preparative C18 reversed-phase column. The mobile phase for elution was a mixture of acetonitrile and D.I. H₂O mixed in different ratios using the programmed gradient (Table 1). The wavelength for detection was set at 225 nm and 280 nm, and the flow rate for elution was 4 ml/min. The major peptide products were characterized by FAB-MS (fast atom bombard mass spectrophotometry) to determine the molecular weight of each peptide. The purity of each peptide was analyzed by analytical RP-HPLC.

### EXAMPLE 2

### Detect the Activities of the Peptides In Vitro

Generally, the in vitro antimicrobial activities of antimicrobial agents were tested using standard NCCLS bacterial inhibition assays, or minimum inhibition concentration (MIC) tests. The MIC value is the lowest concentration of peptide at which the visible growth of test organisms is inhibited and reduced. The test organisms used in the MIC assays are as listed in Table 2.

TABLE 2
Test strains used for MIC determination

| Organism | Source |
|---|---|
| Bacillus. substilis | ATCC 6633 |
| Staphylococcus aureus | ATCC 9144 |
| Staphylococcus epidermidis | ATCC 12228 |
| Staphylococcus aureus | ATCC 29737 |
| Bacillus pumilus | ATCC 14884 |
| Bacillus cereus | ATCC 11778 |
| Pseudomonas aeruginosa | ATCC 29213 |
| Staphylococcus aureus | ATCC 29213 |
| E. coli | ATCC 25922 |

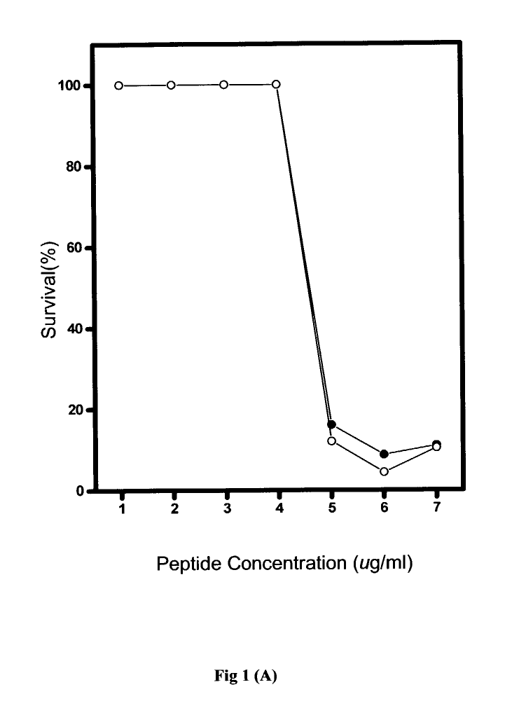

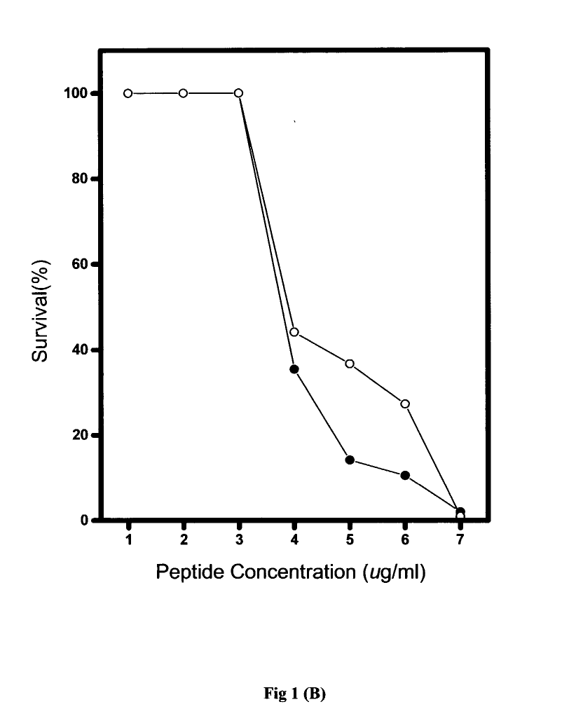

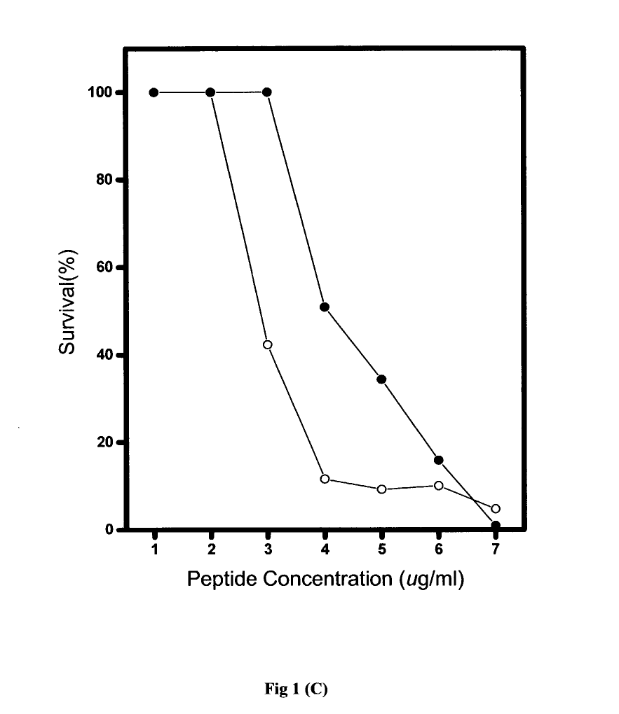

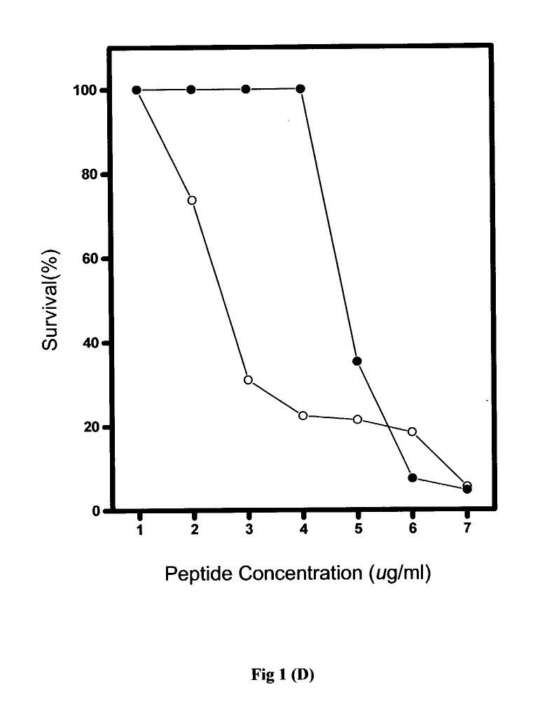

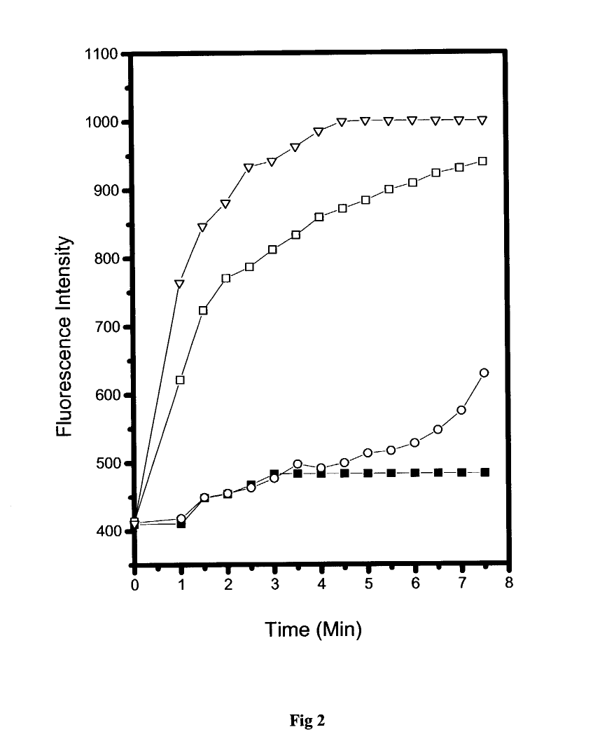

Briefly, overnight cultures of the test organisms were diluted to produce an inoculum containing approximately 10⁵ colonies in Meuller-Hinton broth (MHB). From the peptide stock solution, serial two-dilutions of the peptides in 50 μl volume was prepared in a 96-well microtiter plate, and all wells were subsequently inoculated with the diluted culture of the test organisms. After 18 hours of incubation at 37° C., the results were assayed for turbidity (cell growth). MIC values for each of the peptides are shown in Table 3 and FIG. 1. MIC values were performed three times on different occasions (FIG. 2), and the median values are shown. According to these results, we found that Pac521-530 showed the greatest antimicrobial activity against Gram-positive and Gram-negative bacteria. Furthermore, Pac521-530 showed greater microbicidal activities than Pac 301-310.

TABLE 3
MIC (μg/ml) value for synthetic peptides against E. coli and S.
aureus

| Name | E. coli | S. aureus |
|---|---|---|
| Pac 301 C | >64 | >64 |
| Pac 301 L | 16 | 8 |
| Pac 303 C | >64 | >64 |
| Pac 303 L | 24 | 16 |
| Pac-305 C | >64 | >64 |
| Pac-305 L | 24 | 16 |
| Pac-521 C | 64 | 64 |
| Pac-521 L | 8 | 16 |
| Pac-522 C | 64 | 64 |
| Pac-522 L | 8 | 4 |
| Pac-525 C | 64 | 64 |
| Pac-525 L | 2 | 4 |
| Pac-526 C | 64 | 64 |
| Pac-526 L | 4 | 4 |
| Pac-527 C | 64 | 64 |
| Pac-527 L | 4 | 4 |
| Pac-528 C | 64 | 64 |
| Pac-528 L | 2 | 4 |
| Pac-529 C | 64 | 64 |
| Pac-529 L | 8 | 4 |

* C: cyclic form; L: linear form.

TABLE 4
MIC values for synthetic peptides
MIC g/ml

| | Non Phosphate Buffer | | 1X Phosphate Buffer | |
|---|---|---|---|---|
| Organism | Pac-525 L | Pac-527 L | AP-525 L | Pac-527 L |
| B. substiis | 4 | 4 | 4 | 2 |
| S. epidermidis | 2 | 2 | 2 | 4 |
| S. aureus | 4 | 4 | 8 | 4 |
| B. pumilus | 4 | 2 | 8 | 4 |
| B. cereus | 2 | 4 | 8 | 4 |
| P. aeruginosa | 2 | 4 | 4 | 8 |
| E. coli | 2 | 4 | 8 | 8 |

### EXAMPLE 3

### Membrane Permeabilization Assays

The outer membrane permeabilization activity of the peptide variants was determined by the 1-N-phenylnaphthylamine (NPN) uptake assay, using intact cells of E. coli. NPN performs weak fluorescence in aqueous environment but exhibits strongly in hydrophobic environment. Since NPN is hydrophobic, it provides a direct measurement of the degree of outer membrane permeability. E. coli takes up little or no NPN in a general condition. In the presence of permeabilizer compounds (EDTA, polymyxin B, Neomycin, or antimicrobial peptides), NPN partitions into the bacterial outer membrane and results in an increase in fluorescence. Briefly, use 1 ml of overnight culture to innoculate 50 mls of media and incubate 37° C., shaking. Grow to OD₆₀₀=0.4-0.6. then spin down cells (3500 rpm, 10 min.). Wash and re-suspend in buffer to OD₆₀₀=0.5. Record OD₆₀₀. Add 1 ml of cells (OD₆₀₀=0.5) to cuvette and measure 2-5 seconds. Add 20 ul NPN 0.5 mM (shake to mix) then measure 2-5 seconds. Add 10 ul antibiotic 100X desired final concentration (shake to mix) and measure till the maximal value is reached (1-5 min.). Therefore, fluorescence is varied with the concentration of peptide. The concentration of peptide leading to a 50% of maximal increase in NPN uptake was recorded as the P₅₀. Indeed, all of the peptides were capable of interacting membrane as demonstrated by NPN uptake assay, as shown in Table 5 and FIG. 2.

TABLE 5
The ability to permeablize and promote NPN uptake across outer
membrane of E. coli

| Peptide | P₅₀ (μg/ml) |
|---|---|
| Pac 301 L | 8 |
| Pac 305 L | 16 |
| Pac 309 L | 8 |
| Pac 521 L | 2 |
| Pac 525 L | 2 |
| Pac 529 L | 4 |

### EXAMPLE 4

### Characterization of the Environment of the Trp Resides

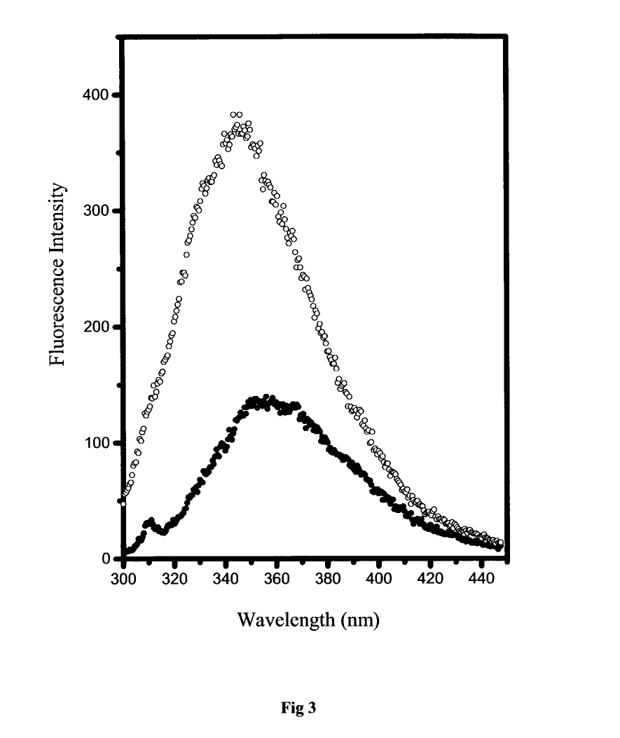

Because of the sensitivity of tryptophan to the polarity of its environment, it has been used for polarity and binding studies. Fluorescence emission spectra were recorded on an LS-55 spectrofluorimeter [Perkin-Elmer] Measurements were performed between 300 and 450 nm at 1 nm increments using a 5 mm quartz cell at 25° C. The excitation wavelength was set to 280 nm with both the excitation and emission slit widths set to 5 nm. In the phosphate buffer, the series of antimicrobial peptides exhibited an emission maximum at 357 nm. In the presence of SDS, they displayed 8 nm blue shift of the emission maximum with a concomitant increase in intensity. The results indicated that the tryptophan side chains had moved into a more hydrophobic environment. The results of this study are shown in FIG. 3.

### EXAMPLE 5

### Secondary Structure of the Peptides Determined by CD Spectroscopy

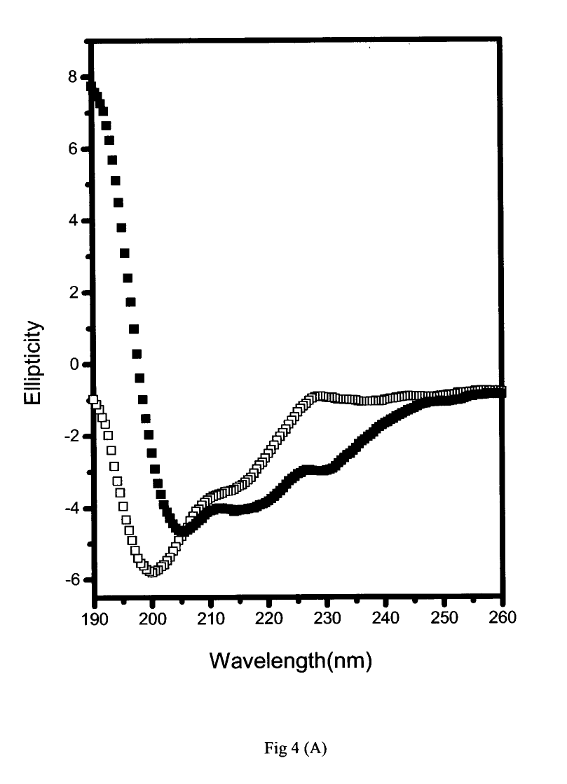

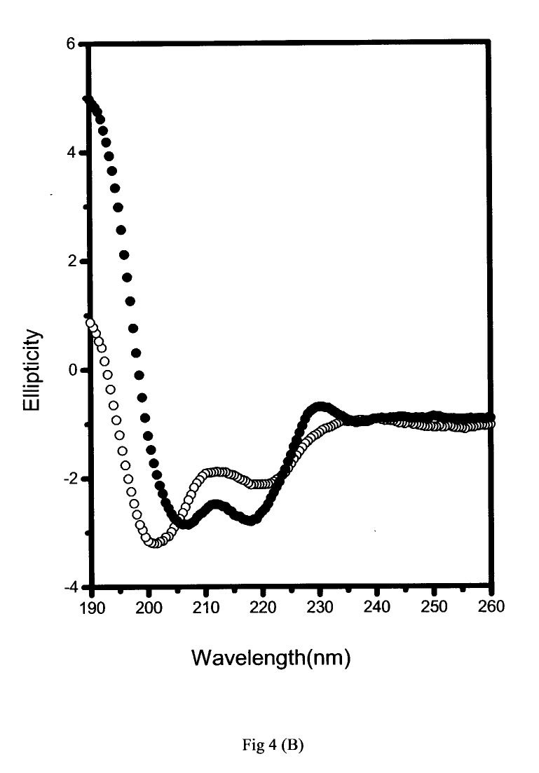

CD spectra were recorded on an AVIV 62DS spectropolarometer after calibration with d-10-camphorsulfonic acid. All measurements were carried out using an 1-mm path-length cuvette at a peptide concentration of 30 μM in 10 mM sodium phosphate buffer of pH 7.2. Far-UV spectra were collected in the range of 190-260 nm using a 0.5-nm stepsize and one second averaging time. In the absence of phospholipid, Pac301-310 and Pac 521-530 are characteristic of unordered structure (Table 6). However, the structures are induced by addition of SDS (FIG. 4).

TABLE 6

| Peptide | conformation in phosphate buffer | conformation in SDS |
|---|---|---|
| Pac 301 C | unordered | unordered |
| Pac 301 L | unordered | slightly ordered |
| Pac 305 C | unordered | unordered |
| Pac 305 L | unordered | slightly ordered |
| Pac 522 C | unordered | unordered |
| Pac 522 L | unordered | slightly ordered |
| Pac 525 C | unordered | unordered |
| Pac 525 L | unordered | slightly ordered |

### EXAMPLE 6

### Erythrocyte Lysis

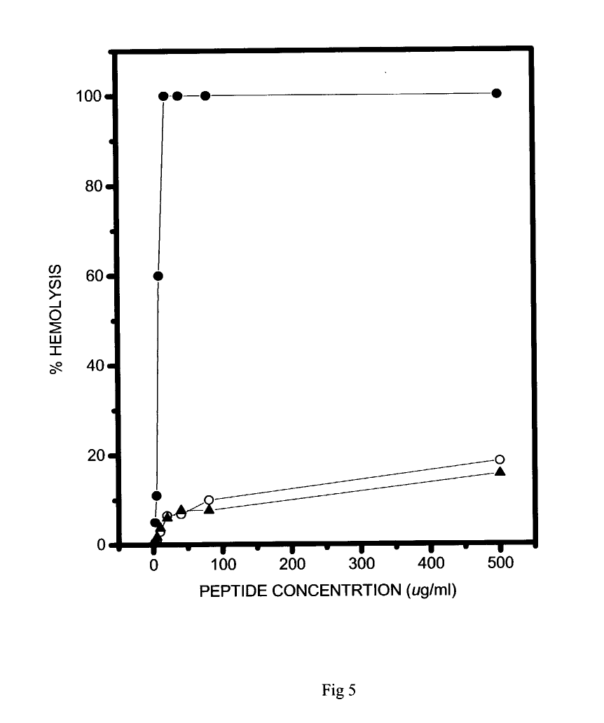

Pac 521-530 were tested for hemolysis against human red blood cells (RBC). The RBCs with EDTA were rinsed 3 times with PBS (800 g, 10 min) and resuspended in PBS. The RBCs were diluted into 10% with phosphate-buffered saline and placed 50 μl into each eppendorf. The peptides that dissolved in PBS were then added to 50 μl of 10% solution of RBCs and incubated for an hour at 37° C. (final RBC concentration, 5% v/v). The samples were centrifuged at 800 g for 10 min at OD₅₄₀. Various concentrations of peptides were incubated with pretreated RBC and the percentage of hemolysis determined. The results showed Pac 525 was substantially less hemolytic against RBC than other antimicrobial peptides (Table 7 and FIG. 5).

TABLE 7

| Peptide | % lysis at 5 μg/ml | % lysis at 50 μg/ml | % lysis at 500 μg/ml |
|---|---|---|---|
| Pac 301 L | 0.85 | 6.8 | 14 |
| Pac 305 L | 0.74 | 7.2 | 15 |
| Pac 524 L | 0.82 | 7.3 | 15 |
| Pac 525 L | 0.81 | 7.2 | 14 |

What is claimed is:

1. A group of cyclic peptide with microbicidal activities can be described by the following formulas:
(A₁X₁A₂)_{N}
wherein:
A₁ represents Arginine, Lysine, Valine, or Isolecine
X₁ represents Trptophan, Phenylalanine, or Proline
A₂ represents Arginine, Lysine, Valine, or Isolecine
N represents 2 or 3
Topology represents cyclic

2. The peptide of claim 1, which is selected from the group consisting of

| | |
|---|---|
| KWRRWI | (SEQ ID NO:1) |
| KWRRWV | (SEQ ID NO:2) |
| KWIKWR | (SEQ ID NO:3) |
| KWVKWI | (SEQ ID NO:4) |
| KWIKWI | (SEQ ID NO:5) |
| KWIKWIKWI | (SEQ ID NO:6) |
| KFIKFIKFI | (SEQ ID NO:7) |
| KWRRWVRWI | (SEQ ID NO:8) |
| IWRVWRRWK | (SEQ ID NO:9) |
| KFRRFVRFI | (SEQ ID NO:10) |
| KPRRPVRPI | (SEQ ID NO:11) |
| KWIRWVRWJ | (SEQ ID NO:12) |

3. The peptides of claim 1 to 2, wherein the peptides consist of linear or cyclic topology.

4. A peptide of claim 1 to 3, wherein the peptide is amidated at the C-terminal amino acid.

5. A peptide of claim 1 to 3, wherein the peptide is acetylated at the N-terminal amino acid.

6. A peptide of claim 1 to 3, wherein the peptide is esterified at the C-terminal amino acid.

7. A peptide of claim 1 to 3 has one or more amino acids altered to a corresponding D-amino acid.

8. A composition comprising a peptide according to claim 1 to 7 in a mixture with a carrier or excipient.

9. A method to inactivate the endotoxin of Gram-negative bacteria comprising the step of administering to a subject in need of such treatment a therapeutically effective amount of antimicrobial peptide according claim 1 to 7.

10. A method to treat or prevent a microbial or viral infection in a subject comprising the step of administering to a subject in need of such treatment a therapeutically effective amount of antimicrobial peptide according claim 1 to 7.

11. A method to inhibit the growth of a microbe comprising the step of administering to a subject in need of such treatment a therapeutically effective amount of antimicrobial peptide according claim 1 to 7.
